(19) United States
(12) Patent Application Publication
(10) Pub. No.: US 2011/0009663 A1
(43) Pub. Date: Jan. 13, 2011

(54) PROCESS FOR PURIFYING AN a-KETO ESTER

(76) Inventors: Wolfgang Wenger, Visp (CH); Anke-Dorothee Braun, Leonberg (DE)

Correspondence Address: HOFFMANN & BARON, LLP, 6900 JERICHO TURNPIKE, SYOSSET, NY, 11791, US

(21) Appl. No.: 12/920,402

(22) PCT Filed: Apr. 8, 2009
(86) PCT No.: PCT/EP09/02602

§ 371 Date: Aug. 31, 2010

Related U.S. Application Data
(60) Provisional application No. 61/095,764, filed on Sep. 10, 2008.

(30) Foreign Application Priority Data
Apr. 14, 2008 (EP) 08007277.0

(51) Int. Cl. C07C 67/48

(52) U.S. Cl. 560/174

(57) ABSTRACT

Process for purifying an α-keto ester by removing secondary and tertiary alcohols from the α-keto ester. In a first step, the α-keto ester to be purified is treated with a carboxylic anhydride and an acid, which is essentially insoluble under the filtration conditions, to esterify the secondary and tertiary alcohols. Subsequent filtration to remove the acid followed by distillation affords the desired purified α-keto ester.

The present invention relates to a process for purifying an α-keto ester as claimed in claim 1.

Various α-keto esters of the general formula

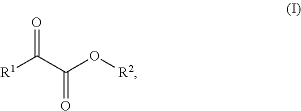

wherein R¹ and R² are as defined below, have already been described in the literature. In general, these compounds are relatively unstable and show keto/enol tautomerism. JP-A-2005336120 and JP-A-2005325050 describe the preparation and/or storage of α-keto esters, whereas the formation of the enol form is to be suppressed.

α-Keto esters are typically prepared by adding a nucleophile to a 1,2-diester. Such reactions are described, for example, in Creary, J. Org. Chem. 1987, 5026-5030, or in Rozen et al., J. Org. Chem. 2001, 496-500. Alternatively, α-keto esters can also be obtained by oxidizing an a-hydroxy ester, for example according to WO 2003/000638.

For the addition of a nucleophile to a 1,2-diester, the nucleophile used may, for example, be a Grignard reagent or an organolithium compound. In this case, the α-keto ester is prepared according to the following reaction scheme

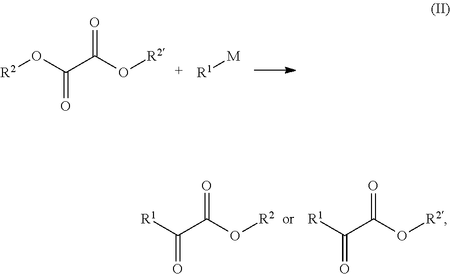

wherein R¹ is alkyl; R², R^{2′} are alkyl or benzyl; and M is Li, MgCl, MgBr or AlR¹₂. An example of such a process is the preparation of methyl 2-oxobutyrate by reaction of dimethyl oxalate with ethylmagnesium chloride. Methyl 2-oxobutyrate is an important building block for the preparation of more complex organic compounds, in particular of pharmaceutical products.

In such addition reactions, secondary and tertiary alcohols of the formula

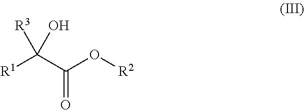

are usually formed as by-products, wherein R¹ is alkyl, R² is alkyl or benzyl and R³ is alkyl or H. These alcoholic by-products are difficult to remove. In particular, on industrial scale the alcohols cannot be removed by the standard methods being customary in this field.

This is true in particular for α-keto esters having a low molecular mass which, by virtue of their relatively low boiling point, would otherwise be highly suitable for distillative purification. Thus, for example, methyl 2-oxobutyrate cannot be purified by distillation since it is not possible to remove the alcoholic by-products in this manner.

JP 09 020 723 discloses a process for purifying an α-keto ester. In this process, the crude product is heated with a strong acid to effect the decomposition of any by-products present. This entails the risk that a part of the desired product is decomposed too, so that a lower yield is obtained.

Therefore, it is an object of the present invention to provide an ecologically and economically advantageous process for purifying an α-keto ester, which process allows secondary and tertiary alcohols to be removed and provides high yields of pure α-keto ester. The process according to the invention should be suitable in particular for use on an industrial scale.

This object is achieved by a process as claimed in claim 1. The process according to the invention allows the purification of an α-keto ester of the formula

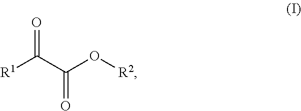

wherein R¹ is a saturated alkyl group with 1-5 carbon atoms, and R² is a saturated alkyl group with 1-5 carbon atoms or is a benzyl group. In this process, the alcoholic by-products resulting from the preparation of the α-keto ester, namely secondary and tertiary alcohols of formula

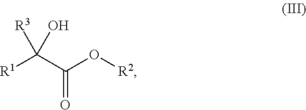

wherein R¹ and R² are as defined above and R³ is hydrogen or a saturated alkyl group with 1-5 carbon atoms, are removed. The process according to the invention comprises the following steps:

(a) treatment of the α-keto ester of formula I, which is to be purified, with a carboxylic anhydride and an acid, which is essentially insoluble under the filtration conditions, for esterifying the secondary and tertiary alcohols of formula III,

(b) filtration of the reaction mixture to remove the acid and

(c) distillation to isolate the purified α-keto ester.

The term “filtration conditions” refers to the external conditions predominating during the filtration, i.e. during step (b) of the process according to the invention. This relates in particular to the temperature and the pressure at which the filtration is carried out. The filtration conditions are furthermore characterized by components present in the reaction mixture prior to the filtration, in particular any solvents present.

By treatment with the carboxylic anhydride under acidic conditions, the secondary and tertiary alcohols are converted into the corresponding diester according to the following reaction scheme

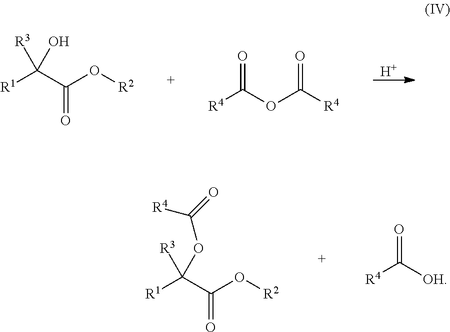

In contrast, the α-keto ester is inert to these reagents and remains unchanged. The esterification under acidic conditions is advantageous, in particular when tertiary alcohols are to be removed, since these are relatively inert. The esterification is preferably carried out at a temperature of 20-100° C., in particular at 40-80° C., for example at about 50° C., and at atmospheric pressure under protective gas.

After esterification, the acid, which is essentially insoluble under the filtration conditions, is separated off by filtration. This permits a rapid and inexpensive removal of the acid and supersedes an extraction, which would require the use of solvents, thus leading to additional costs. Moreover, a filtration is, in particular on an industrial scale, considerably less complicated than an extraction, and less product is lost. Last but not least, the acid can be isolated in a simple manner and, if desired, re-used.

Since the boiling points of the α-keto ester and the esterified secondary and tertiary alcohols, i.e. the corresponding diesters, differ considerably, the different products can subsequently be separated by distillation, and the α-keto ester can be obtained in pure form.

The process according to the invention permits a rapid and efficient purification of the α-keto ester and affords a product having a high degree of purity. The degree of purity of the α-keto ester obtained in this manner is preferably at least 94%, in particular at least 97%, ideally at least 98%. The process steps are neither dangerous nor complicated and are inexpensive.

In a preferred embodiment, the acid which is essentially insoluble under the filtration conditions is a solid at the filtration temperature. The term “filtration temperature” refers to the temperature at which the reaction mixture is filtered. For example, acidic polysilicates are employed as acids. Suitable acidic polysilicates are, for example, amorphous polysilicates of the allophane type; chain polysilicates of the hormite type, such as, for example,
polygorskite;

two-layer polysilicates of the kaolin type, such as, for example,
kaolinite (Al₂(OH)₄[Si₂O₅]), and
halloysite (Al₂(OH)₄[Si₂O₅]×2 H₂O);

three-layer polysilicates of the smectite type, such as, for example,
sauconite (Nao_{0.3}Zn₃(Si,Al)₄O₁₀(OH)₂×4 H₂O),
saponite ((Ca₂,Na)_{0.3}(Mg,Fe²⁺)₃ (Si,Al)₄O₁₀(OH)₂×4 H₂O),
montmorillonite (M⁺_{0.3}(Al,Mg)₂Si₄O₁₀(OH)₂×n H₂O), wherein M⁺ in natural
montmorillonite denotes one or more of the cations Na⁺, K⁺, Mg²⁺ and Ca²⁺,
vermiculite ((Mg,Fe²⁺,Al)₃(Al,Si)₄O₁₀(OH)₂×4 H₂O),
nontronite (Na_{0.3}Fe₂³⁺ (Si,Al)₄O₁₀(OH)₂×4 H₂O), and
hectorite (Na_{0.3}(Mg,Li)₃Si₄O₁₀(F,OH)₂);

three-layer polysilicates of the illite type; and

polysilicates having variable layers of the chlorite type and tectopolysilicates, such as zeolites, preferably of type Y in its H-form.

If required, such acidic polysilicates can be activated by treatment with acid and/or by treatment with a metal salt solution and/or by drying, and in the case of zeolites preferably by ion exchange and/or by heating.

Particularly suitable is montmorillonite K10 (for example from Süd-Chemie), being a sheet silicate of the smectite type, which can act both as Brönsted and as Lewis acid. Montmorillonite K10 is inexpensive, non-toxic and not dangerous and is accordingly suitable in particular for carrying out the process according to the invention on an industrial scale.

In another preferred embodiment, the acid is attached to a carrier, whereas the carrier being attached to the acid is a solid at filtration temperature. Suitable carriers are, for example, polystyrenes, polyethylene glycols, polyacrylamides, silicon dioxide, controlled pore glass (CPG) or resin beads. Since the combination compound of acid and carrier is a solid at the filtration temperature, it can be filtered off and removed from the reaction mixture very easily.

In an also preferred embodiment, the substituent R¹ of the α-keto ester is a straight-chain or branched alkyl group with 1-3 carbon atoms. The process according to the invention can be used in particular for purifying methyl 2-oxobutyrate. The advantages of the present invention are particularly evident in the case of methyl 2-oxobutyrate since the corresponding secondary and tertiary alcohol by-products cannot be esterified, for example, with acetic anhydride alone or in combination with N,N-dimethylaminopyridine (DMAP).

Preferably, the carboxylic anhydride used is acetic anhydride. Compared to other carboxylic anhydrides, acetic anhydride is relatively cheap and can be obtained in large amounts. Moreover, with respect to the so called “Atom Economy”, the use of acetic anhydride is particularly advantageous since the total amount of waste can be kept at a minimum. Preferably, acetic anhydride is employed in an amount of less than 50 percent by weight, based on the amount of the crude α-keto ester product, for example in an amount of 2-25 percent by weight, in particular in an amount of 5-15 percent by weight.

The acid employed in step (a) is preferably used in catalytic amounts. In this manner, the process costs can further be reduced, and the risk of any unwanted side reactions during the esterification is kept at a minimum. The acid is preferably employed in an amount of less than 20 percent by weight, based on the amount of the crude α-keto ester product, more preferably in an amount of less than 10 percent by weight, for example in an amount of 2-5 percent by weight.

It is particularly advantageous, if the acid is recycled after filtration, i.e. after step (b). In this manner, the costs of purifying the α-keto ester can be reduced even further. Moreover, there are less costs for the disposal of the acid.

The present invention furthermore relates to the use of montmorillonite K10 for purifying an α-keto ester, in particular methyl 2-oxobutyrate. Here, montmorillonite K10 is used in combination with a carboxylic anhydride, in particular with acetic anhydride, to esterify any alcoholic by-products present, so that they can be removed afterwards by distillation.

The present invention is now illustrated in more detail by the examples below. Example 1 relates to a process according to the invention, whereas examples 2 and 3 describe esterification experiments under different reaction conditions.

### EXAMPLE 1

In a 1 L round-bottom flask, 737.2 g of methyl 2-oxobutyrate to be purified, with a methyl 2-oxobutyrate content of about 62%, 69.5 g of acetic anhydride and 23.9 g of montmorillonite K10 are stirred on a rotary evaporator at 50° C. for one hour. The reaction mixture is then filtered through a glass suction filter. Distillation at a head temperature of 48-50° C. and a pressure of 15 mbar affords 360 g of methyl 2-oxobutyrate having a methyl 2-oxobutyrate content of more than 98%.

The results of the product analysis by gas chromatography are summarized in table 1.

TABLE 1

| | Relative | Relative | Relative |
|---|---|---|---|
| | content of | content of | content of |
| | methyl 2-oxobutyrate | secondary alcohol | tertiary alcohol |
| Crude | 92.50% | 3.97% | 3.53% |
| product | | | |
| Filtered | 99.97% | 0.03% | — |
| product* | | | |

*Before distillation.

### EXAMPLE 2 (Comparative Example)

5.0 g of methyl 2-oxobutyrate to be purified and 0.5 g of acetic anhydride are stirred for one hour at 40° C., 60° C. and 80° C., respectively. The reaction mixture is then filtered through a glass suction filter.

The reaction mixture is analyzed by gas chromatography. The results are summarized in Table 2.

TABLE 2

| | Relative | Relative | Relative |
|---|---|---|---|
| | content of | content of | content of |
| | methyl 2-oxobutyrate | secondary alcohol | tertiary alcohol |
| Crude | 91.72% | 5.58% | 2.75% |
| product | | | |
| 40° C. | 91.50% | 5.50% | 3.00% |
| 60° C. | 91.61% | 5.57% | 2.82% |
| 80° C. | 91.57% | 5.52% | 2.91% |

### EXAMPLE 3 (Comparative Example)

5.0 g of methyl 2-oxobutyrate to be purified, 0.5 g of acetic anhydride and 25 mg of DMAP are stirred for one hour at 40° C., 60° C. and 80° C., respectively. The reaction mixture is then filtered through a glass suction filter.

The reaction mixture is analyzed by gas chromatography. The results are summarized in Table 3.

TABLE 3

| | Relative | Relative | Relative |
|---|---|---|---|
| | content of | content of | content of |
| | methyl 2-oxobutyrate | secondary alcohol | tertiary alcohol |
| Crude | 91.72% | 5.58% | 2.75% |
| product | | | |
| 40° C. | 90.21% | 5.80% | 3.99% |
| 60° C. | 91.54% | 4.74% | 3.72% |
| 80° C. | 91.30% | 4.84% | 3.86% |

1. A process for purifying an α-keto ester of formula 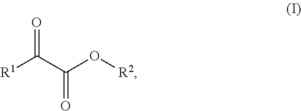
wherein R¹ is a saturated alkyl group with 1-5 carbon atoms, and R² is a saturated alkyl group with 1-5 carbon atoms or is a benzyl group,
having a content of secondary and tertiary alcohols of formula 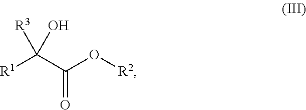
wherein R¹ and R² are as defined above and R³ is hydrogen or a saturated alkyl group with 1-5 carbon atoms,
characterized in that
(a) the α-keto ester of formula I, which is to be purified, is treated with a carboxylic anhydride and an acid, said acid being an acidic polysilicate, which is essentially insoluble under the filtration conditions, for esterifying the secondary and tertiary alcohols of formula III,
(b) the reaction mixture is filtered to remove the acid, and
(c) the purified α-keto ester is distilled for isolation.

2. The process of claim 1, wherein the acid is a solid at filtration temperature.

3. The process of claim 1, wherein the acid is attached to a carrier and the carrier being attached to the acid is a solid at filtration temperature.

4. The process of claim 1, wherein the acid is montmorillonite K10.

5. The process of claim 1, wherein R¹ is a straight-chain or branched alkyl group with 1-3 carbon atoms.

6. The process of claim 1, wherein the α-keto ester is methyl 2-oxobutyrate.

7. The process of claim 1, wherein the carboxylic anhydride is acetic anhydride.

8. The process of claim 1, wherein the acid is recycled after filtration.

9. The process of claim 1, wherein the acid is employed in a catalytic amount.

10. Use of montmorillonite K10 in combination with a carboxylic anhydride for purifying methyl 2-oxobutyrate.
